(19) United States
(12) Patent Application Publication
(10) Pub. No.: US 2022/0005603 A1
(43) Pub. Date: Jan. 6, 2022

(54) DE-NOISING TASK-SPECIFIC ELECTROENCEPHALOGRAM SIGNALS USING NEURAL NETWORKS

(71) Applicant: X Development LLC, Mountain View, CA (US)

(72) Inventors: Garrett Raymond Honke, Mountain View, CA (US); Pramod Gupta, Mountain View, CA (US); Irina Higgins, London (GB)

(21) Appl. No.: 16/921,224

(22) Filed: Jul. 6, 2020

(51) Int. Cl. G16H 50/20; G06N 3/08

(52) U.S. Cl. CPC G16H 50/20; G06N 3/08

(57) ABSTRACT

Methods, systems, and apparatus, including computer programs encoded on computer storage media, for training an auto-encoder to de-noise task specific electroencephalogram (EEG) signals. One of the methods includes training a variational auto-encoder (VAE) including to learn a plurality of parameter values of the VAE by applying, as first training input to the VAE, training data, the training data comprising electroencephalogram (EEG) data representing brain activities of individual persons when performing different tasks; and after the training, adapting the VAE for a specific task by applying, as second training input to the VAE, adaptation data, the adaptation data comprising task-specific EEG data representing brain activities of individual persons when performing the specific task.

### BACKGROUND

This specification relates to using neural networks to process electroencephalogram (EEG) data.

Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

An electroencephalogram (EEG) is a measurement of scalp-level electrical activity projected from a person's brain (among other sources). EEG measures the electrical activity of large, synchronously firing populations of neurons in the brain with electrodes placed on the scalp.

### SUMMARY

In general, innovative aspects of the subject matter described in this specification can be embodied in methods that include the actions of training an auto-encoder to de-noise task specific electroencephalogram (EEG) signals. The actions include training a variational auto-encoder (VAE) including to learn a plurality of parameter values of the VAE by applying, as first training input to the VAE, training data, the training data comprising electroencephalogram (EEG) data representing brain activities of individual persons when performing different tasks, where the VAE is configured to receive as input EEG data, process the EEG data to determine a latent representation of the EEG data, and to process the latent representation to generate a reconstruction of the EEG data; and after the training, adapting the VAE for a specific task by applying, as second training input to the VAE, adaptation data, the adaptation data comprising task-specific EEG data representing brain activities of individual persons when performing the specific task, where adapting the VAE includes adjusting learned parameter values of the VAE by optimizing an adaptation objective function that depends at least on a quality of the reconstructions of the task-specific EEG data.

In some implementations, the actions include training the VAE using a de-noising auto-encoder training technique. In some implementations, the actions include: after the adaptation, providing data specifying the trained VAE for deployment in an EEG diagnosis system to determine mental health conditions of individual persons based on processing EEG data. In some implementations, the EEG diagnosis system includes one or more classification models or one or more prediction models. In some implementations, the specific task includes a reinforcement learning reward task. In some implementations, the VAE is a convolutional disentangling VAE, and the actions include: receiving a plurality of first training inputs, and, for each first training input: processing the first training input using the VAE to determine a latent representation that includes a plurality of latent factors and to generate a reconstruction of the first training input in accordance with current values of the parameters of the VAE; and adjusting current values of the parameters of the VAE by optimizing a training objective function that depends on a quality of the reconstruction and also on a degree of independence between the latent factors in the latent representation of the first training input. In some implementations, some or all of the plurality of first training inputs are unlabeled EEG training inputs.

Other implementations of this aspect include corresponding systems, apparatus, and computer programs, configured to perform the actions of the methods, encoded on computer storage devices.

The subject matter described in this specification can be implemented in particular embodiments so as to realize one or more of the following advantages. Using the described techniques, the system can first train a variational auto-encoder (VAE) broadly across different EEG data which is publicly available or otherwise easily obtainable in massive volumes. The trained VAE can then be used to efficiently adapt for a specific type of cognitive task using orders of magnitude less data than was used to train the VAE. For example, while training the VAE may utilize hours of observations of EEG signal segments collected from a diverse range of tasks, adapting the VAE for a specific task may require merely a few minutes of observations of task-specific EEG signal segments.

This two-stage process also enables technological use cases that were previously not possible. First, high quality (e.g., accurate, cleaned, or both) EEG signals can be generated from input EEG signals for cognitive tasks under certain categories for which EEG data is expensive or difficult to collect for use in effective training of a de-noising ML model. Second, because a significantly reduced amount of task-specific EEG data is needed, the adaptation process is much less computationally intensive than the training process. The adaptation process therefore can be performed by consumer hardware of end users, e.g., a desktop or laptop computer, rather than being performed in a datacenter.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example variational auto-encoder (VAE) system.

FIG. 2 is a flow diagram of an example process for training a variational auto-encoder.

FIG. 3 shows an example electroencephalogram (EEG) diagnosis system.

FIG. 4 depicts a schematic diagram of a computer system that may be applied to any of the computer-implemented methods and other techniques described herein.

Like reference numbers and designations in the various drawings indicate like elements.

### DETAILED DESCRIPTION

This specification describes a system implemented as computer programs on one or more computers in one or more locations that trains a variational auto-encoder neural network to de-noise task specific electroencephalogram (EEG) signals.

An electroencephalogram (EEG) is a measurement that detects electrical activity projected from a person's brain to their scalp. EEG measures the electrical activity of large, synchronously firing populations of neurons in the brain with electrodes placed on the scalp. The EEG signals, even under controlled conditions, may contain significant noise, e.g., due to biological and/or electrical sources. The propensity for noise is further increased outside of a well-controlled laboratory environment. Accordingly, machine learning (ML)-based noise reduction may be particularly beneficial in providing usable EEG data in real-time under real world conditions (e.g., outside of a well-controlled environment). For example, EEG data may be collected and provided under such conditions to another system for use in diagnosis of individual psychopathology based on properties of the cleaned EEG signal.

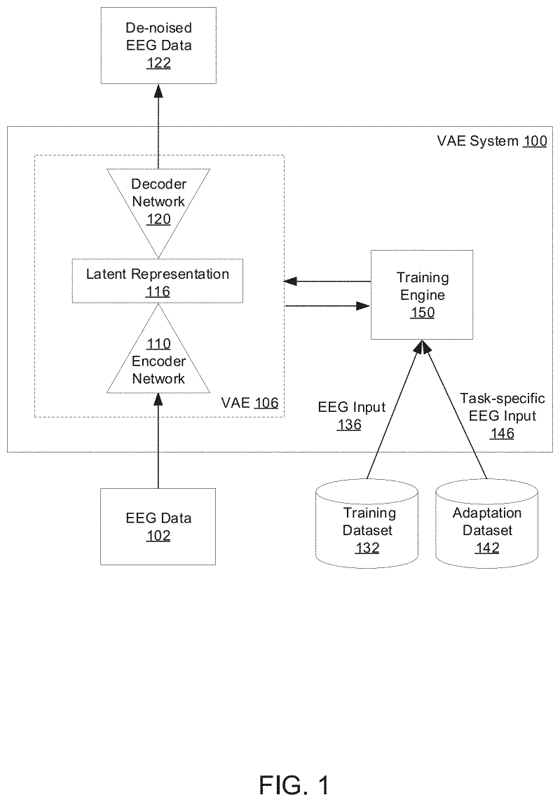

FIG. 1 shows an example variational auto-encoder (VAE) system 100. The VAE system 100 is an example of a system implemented as computer programs on one or more computers in one or more locations, in which the systems, components, and techniques described below can be implemented.

The VAE system 100 includes a variational auto-encoder 106 and a training engine 150. The variational auto-encoder 106 can receive as input EEG data 102, e.g., an EEG signal segment, and process that input to generate a de-noised, e.g., cleaned, reconstruction 122 of the EEG data. In other words, the VAE 106 can be used to clean the EEG data 102 to reduce signal noise, thereby improving usability of the EEG data for subsequent analysis.

The VAE 106 includes an encoder network 110 and a decoder network 120 that are each a respective neural network that can include one or more neural network layers, including one or more fully connected layers, one or more convolutional layers, and/or one or more recurrent layers.

The encoder network 110 is configured to receive the input EEG data 102 and process the EEG data 102 in accordance with a plurality of encoder network parameters to generate a latent representation 116 based on the EEG data 102.

In some implementations, the latent representation 116 is a lower-dimension, i.e., compressed, version of the input EEG data 102.

In some implementations, the latent representation 116 includes a set of latent factors and can represent features of the input EEG data 102. A latent factor is any value that is defined by the outputs of the encoder network 110 based on processing the input EEG data 102.

In some such implementations, the encoder network 110 generates an output that, for each latent factor, parameterizes a distribution, e.g., a Gaussian distribution, over a set of possible values for the latent factor and samples a value for the latent factor from the distribution.

The decoder network 120 is configured to receive the latent representation 116 and process the latent representation 116 in accordance with a plurality of decoder network parameters to generate a de-noised reconstruction 122 of the EEG data 102.

The training engine 150 in the system 100 trains the VAE 106 to determine trained values of the parameters, including the plurality of encoder network parameters and the plurality of decoder network parameters, from initial values of the parameters using an iterative training process. At each iteration of the training process, the training engine 150 determines an update to the current network parameter values and then applies the update to the current network parameter values.

A present limitation of training the VAE 106 to effectively de-noise EEG data is that training the VAE typically requires large, labeled EEG training datasets. In addition, applying the VAE in different use cases, e.g., to denoise EEG data under different task categories or different outcome categories, usually requires different training datasets. Ideally, for a given use case, the training system needs multiple thousands of observations of labeled EEG signal segments in order to train the VAE to generate cleaned reconstructions of input EEG signals. This amount of data is expensive and cumbersome to obtain and curate in general, and there are many use cases for which this volume of training data is impractical or impossible to obtain.

Thus, to improve the effectiveness of the training, the implementations of the present disclosure train the neural network using a two-stage process: training and adaptation. In particular, the training engine 150 first trains the variational auto-encoder (VAE) 106 using a diverse set of training EEG inputs 136 that are publicly available or otherwise easily obtainable in massive volumes. For example, each EEG input 136 included in the training dataset 132 can be an EEG signal segment. For example, each EEG input 136 can be derived from electroencephalogram (EEG) data representing brain activities of individual persons when performing different tasks. Such EEG data may be collected from a range of different sources, e.g., different user groups, experimental conditions, or devices, may be unlabeled, or may be un-preprocessed, e.g., unfiltered, unamplified, or unsynchronized.

After the training, the training engine 150 adapts the trained VAE for a specific task using an adaptation dataset 142 drawn from a particular task of interest, e.g., a reinforcement learning reward task, a motor imagery task, or a visual stimulus processing task. As a particular example of a reinforcement learning reward task, a gambling task is a task typically used to detect neurological reward system dysfunction where users receive a small reward for making winning bets and a penalty for incorrect bets. The adaptation dataset 142 includes a plurality of task-specific EEG segments time locked to the presentation of the reward signal 146 representing brain activities of individual persons when performing the specific task. The VAE system 100 can access multiple adaptation datasets each dedicated to a different type of cognitive task. These datasets, for example, can be stored at one or more memory devices accessible to the system 100. By repeatedly using the training engine 150 to train an instance of the VAE on a respective adaptation dataset, the VAE system 100 can obtain multiple VAEs each adapted for de-noising EEG signals associated with a particular type of cognitive task.

Generally the data used for the adaptation process can be orders of magnitude smaller than data used for the training process. In some implementations, the training dataset 132 includes billions of EEG inputs 136, while each adaptation dataset 142 includes merely a few thousand task-specific EEG inputs 146.

Once the two-stage process has completed, the system can provide data specifying the trained VAE for deployment in an EEG system useful for applications such as brain-computer interfacing, cognitive state detection, biometrics or psychopathology diagnoses of individual persons based on processing the cleaned EEG signal generated by using the VAE. For example, the EEG diagnosis system can implement one or more classification model, or one or more prediction models

Instead of or in addition to providing the data specifying the trained VAE, the system 100 can use the trained VAE 106 to process new EEG data 102 and generate de-noised reconstruction 112 of the new EEG data.

It should be noted that, while the description in this specification largely relates to effectively obtaining task-specific de-noising VAEs, the described techniques can also be used for adapting a VAE that has been trained broadly across diverse EEG data for any of a variety of other use cases. For example, the techniques can be similarly used to adapt a broadly trained VAE for use in processing EEG data collected for a specific age group, e.g., over 40 or under 20, a specific gender group, e.g., male or female, or a specific type of task outcome, e.g., winning, losing, arousing, or neutral outcome.

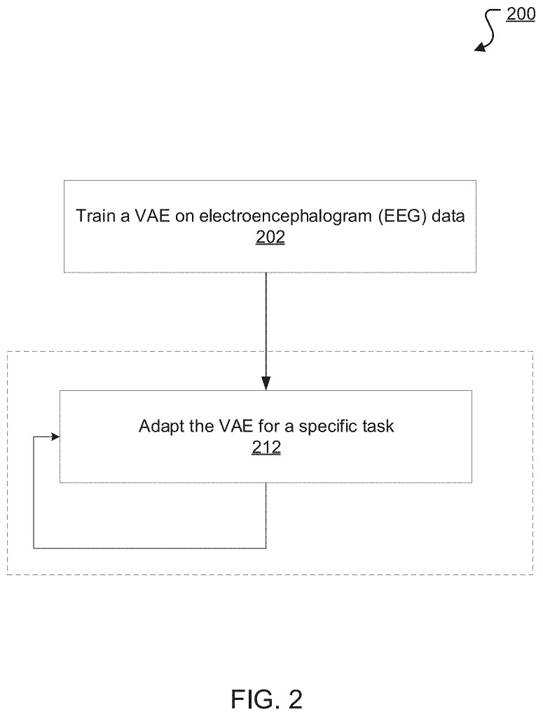

FIG. 2 is a flow diagram of an example process 200 for training a variational auto-encoder. For convenience, the process 200 will be described as being performed by a system of one or more computers located in one or more locations. For example, a system, e.g., the VAE system 100 of FIG. 1, appropriately programmed in accordance with this specification, can perform the process 200.

The system trains a variational auto-encoder (VAE) (202) by applying a set of training data as training input to the VAE. As described above, the training data can be derived from electroencephalographic (EEG) data representing brain activities of individual persons when performing different tasks.

At each training iteration, the system can receive a mini-batch of training inputs. A mini-batch generally includes a fixed number of training inputs, e.g., 16, 64, or 256. For example, each training input can include a respective EEG signal segment. For each training input, the system uses an encoder of a VAE, e.g., the encoder network 110 of the VAE 106 of FIG. 1, to generate a latent representation of the training input and process the latent representation using a decoder of the VAE, e.g., the decoder network 120 of the VAE 106 of FIG. 1, to generate a reconstruction of the training input. The VAE generates the reconstruction based on the current values of the parameters of VAE. At the end of each training iteration, the system applies respective updates to the current values of the parameters of the VAE by optimizing a VAE training objective function, e.g., based on computing a gradient of the VAE training objective function with respect the VAE parameters and using a gradient descent optimization technique, e.g., an RMSprop or Adam technique.

In some implementations, the VAE training objective function includes a first term which measures a quality of the reconstruction in an output of VAE, i.e., relative to the input, and a second term which measures a match between the similarity of a given distribution, a Gaussian distribution, and the distributions parameterized by the encoder network output from which the latent factors are sampled. In this way, the system trains the encoder network of the VAE to generate latent representations that represent features of the input EEG signal segments and that allow for easy sampling and interpolation.

In some implementations, the system trains the VAE using a β-VAE training technique. β-VAE is a modification of the variational autoencoder (VAE) framework that introduces an adjustable hyperparameter β to the original VAE objective function. In such implementations, the VAE training objective function includes a first term which measures of a quality of reconstruction, and a scaled second term which measures of degree of independence between latent factors generated by the VAE. By appropriately setting the value of the hyperparameter that is used to scale the second term, e.g., to a value greater than one, the latent representations generated by the encoder network can become disentangled latent representations. β-VAE is described in more detail in Irina Higgins, Loic Matthey, Arka Pal, Christopher Burgess, Xavier Glorot, Matthew Botvinick, Shakir Mohamed, and Alexander Lerchner. β-VAE: Learning basic visual concepts with a constrained variational framework. In this way, the system trains the VAE so that the encoder network generates disentangled representations and the decoder network generates accurate reconstructions of input EEG signal segments based on the representations generated by encoder network.

In some implementations, the system trains the VAE using a de-noising autoencoder training technique which first computes, e.g., by means of a stochastic mapping, a corrupted version of a training input and then trains the VAE to reconstruct the training input from the corrupted version of training input. Such a technique is described in Pascal Vincent, Hugo Larochelle, Yoshua Bengio, Pierre-Antoine Manzagol. Extracting and Composing Robust Features with Denoising Autoencoders. Using this technique may, in some circumstances, improve the robustness of the VAE against noise in input EEG signal segments while resulting in the VAE generating meaningful reconstructions of the input EEG signal segments.

The system proceeds to adapt the VAE for a specific task (212) after the training has terminated, e.g., after a predetermined number of training iterations have been performed or after the gradient of the VAE training objective function has converged to a specified value.

The system can do this by applying a set of adaptation data as training input to the VAE. As described above, while the training data can include EEG data collected across diverse tasks, the adaptation data is specifically drawn from a particular task of interest, e.g., a reinforcement learning reward task, a motor imagery task, or a visual stimulus processing task. In other words, the adaptation data include task-specific EEG data representing brain activities of individual persons when performing the particular task.

During adaptation, the system fine-tunes the learned values of the VAE parameters by retraining with respect to the task-specific adaptation dataset. In brief, for each training input included in the adaptation dataset, the system uses the encoder of the VAE to generate a latent representation of the training input and process the latent representation using the decoder of the VAE to generate a reconstruction of the training input. The system then applies respective updates to the current values of the parameters of the VAE by optimizing a VAE adaptation objective function, e.g., based on computing a gradient of the VAE adaptation objective function with respect the VAE parameters and using a gradient descent optimization technique, e.g., an RMSprop or Adam technique. Example components of the VAE adaptation objective function are similarly described above with reference to step 202. In this way, the VAE parameter values learned during training process are adjusted so that they are adapted to the particular task of interest.

The system can repeatedly perform the step 212 to adapt multiple instances of the VAE for different cognitive tasks. That is, the system can train each of multiple instances of the VAE on a respective adaptation dataset and thereby adapt each instance of the VAE for a particular type of cognitive task. The system can do this in parallel, asynchronously, or in a decentralized manner.

In general, after the adaptation, the multiple instances of the VAE can each output cleaned signals, e.g., EEG signals that have a reduced signal-to-noise ratio as compared with input signals, from input EEG signals collected for a specific type of cognitive task. The cleaned EEG signals can be used as input features to a machine learning model to determine a psychological and/or motivational state of the user. A particular example of using the cleaned EEG signals to determine whether a patient is or will be experiencing a mental health condition is described below. Specifically, in this example, the system provides, e.g., through a wired or wireless connection, data specifying the trained VAEs for deployment in an EEG diagnosis system that is configured to determine psychopathology diagnoses of individual persons based on processing the cleaned EEG signals.

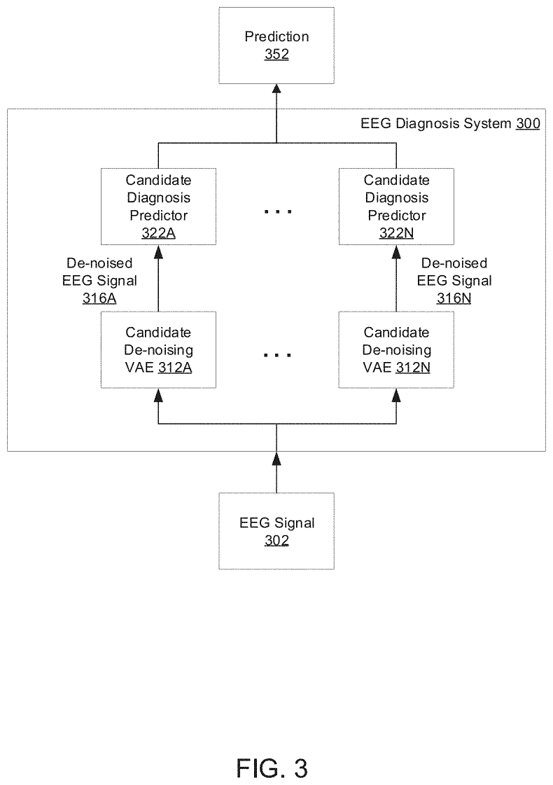

FIG. 3 shows an example electroencephalogram (EEG) diagnosis system 300. The system 300 is an example of a system implemented as computer programs on one or more computers in one or more locations, in which the systems, components, and techniques described below are implemented.

In the example of FIG. 3, the system includes a plurality of diagnosis predictors, e.g., candidate diagnosis predictors 322A-N, each associated with a respective de-noising VAE, e.g., candidate de-noising VAEs 312A-N. However, the diagnosis predictors need not have a one-to-one correspondence with de-noising VAEs and there may be a different number of diagnosis predictors, or a different number of de-noising VAEs. Each de-noising VAE can be a VAE that has been first trained on diverse EEG data and then adapted specifically for a given task category by using the techniques described above. Each diagnosis predictor can be configured to process and analyze EEG data under a different task category. Each diagnosis predictor can have any appropriate machine learning model architecture. For example, the diagnosis predictor may be a neural network model, a random forest model, a support vector machine (SVM) model, a linear model, or a combination thereof.

As such, the EEG diagnosis system 300 can receive EEG data collected for any of a variety of cognitive tasks and to generate any of a variety of kinds of classification output based on the input.

In particular, to generate such output, the system 300 first uses a VAE to de-noise the input EEG signal 302 and thereafter process the de-noised EEG signal using a diagnosis predictor to generate a corresponding prediction 352. In some implementations, the system can select, from a plurality of candidate de-noising VAEs 312A-N and based on the task category of the input EEG signal 302, a selected VAE (e.g., candidate de-noising VAE 312A) for use in processing the input to generate a de-noised reconstruction of the EEG signal (e.g., de-noised EEG signal 316A). In some implementations, the system can similarly select a selected diagnosis predictor (e.g., candidate diagnosis predictor 322A) for use in processing the denoised reconstruction of the EEG signal to generate an output that specifies the prediction 352 of a classification of the EEG signal 302.

For example, if the input to the EEG diagnosis system 300 include EEG signals collected from an individual person performing one or more executive function tasks, e.g., reinforcement learning reward task, motor imagery task visual stimulus processing task, the output generated by the EEG diagnosis system 300 for the input may be scores for each of a set of mental disorder categories, e.g., attention deficit disorder, Major Depressive Disorder (MDD) and affective disorder, Parkinson's disease, and Schizophrenia, with each score representing an estimated likelihood that the individual person performing the tasks is a patient with the corresponding mental disorder.

As another example, if the input to the EEG diagnosis system 300 include EEG signals collected from a patient with a particular mental disorder performing one or more executive function tasks, the output generated by the EEG diagnosis system 300 for the input may be scores for each of a set of disease stages, e.g., mild, moderate, and prodromal, with each score representing an estimated likelihood that the patient is at the corresponding stage of the particular mental disorder.

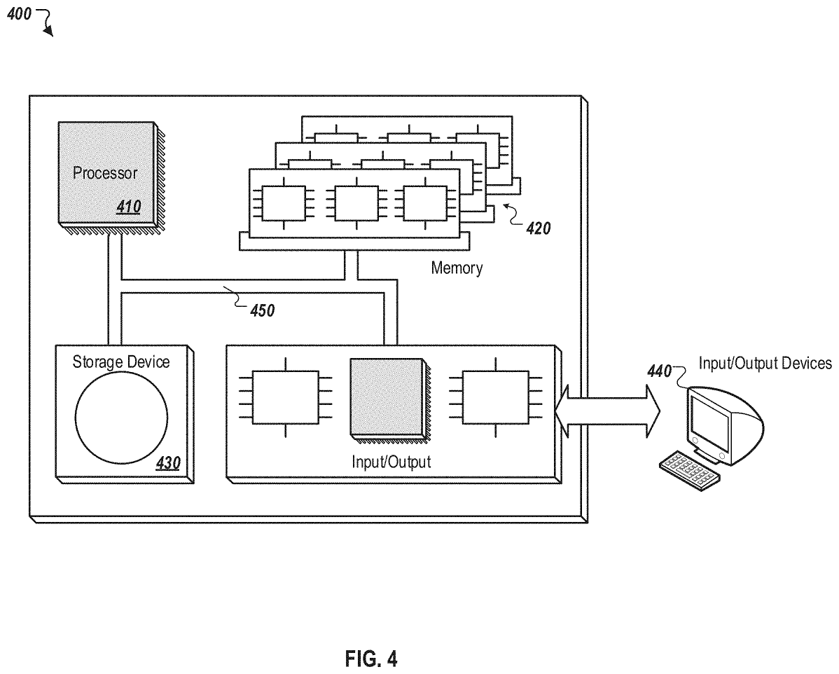

FIG. 4 is a schematic diagram of a computer system 400. The system 400 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 400) and their structural equivalents, or in combinations of one or more of them. The system 400 is intended to include various forms of digital computers, such as laptops, desktops, workstations, personal digital assistants, servers, blade servers, mainframes, and other appropriate computers, including vehicles installed on base units or pod units of modular vehicles. The system 400 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

While the present disclosure is described in the context of a psychological diagnostic system, it is understood that the techniques and processes described herein are applicable outside of this context. For example, the techniques and processes described herein may be applicable to other types of diagnostic machine learning systems including, but not limited to, medical diagnostic systems, computer software diagnostic (debugging) systems, computer hardware diagnostic systems, or quality assurance (e.g., in manufacturing) diagnostic systems.

What is claimed is:

1. A method of training an auto-encoder to de-noise task specific electroencephalogram (EEG) signals, the method comprising:
training a variational auto-encoder (VAE) including to learn a plurality of parameter values of the VAE by applying, as first training input to the VAE, training data, the training data comprising electroencephalogram (EEG) data representing brain activities of individual persons when performing different tasks, wherein the VAE is configured to receive as input EEG data, process the EEG data to determine a latent representation of the EEG data, and to process the latent representation to generate a reconstruction of the EEG data; and
after the training, adapting the VAE for a specific task by applying, as second training input to the VAE, adaptation data, the adaptation data comprising task-specific EEG data representing brain activities of individual persons when performing the specific task, wherein adapting the VAE comprises adjusting learned parameter values of the VAE by optimizing an adaptation objective function that depends at least on a quality of the reconstructions of the task-specific EEG data.

2. The method of claim 1, wherein adapting the VAE for the specific task comprises:
training the VAE using a de-noising auto-encoder training technique.

3. The method of claim 1, further comprising, after the adaptation:
providing data specifying the trained VAE for deployment in an EEG diagnosis system to determine mental health conditions of individual persons based on processing EEG data.

4. The method of claim 3, wherein the EEG diagnosis system comprises one or more classification models or one or more prediction models.

5. The method of claim 1, wherein the specific task comprises a reinforcement learning reward task.

6. The method of claim 1, wherein the VAE is a convolutional disentangling VAE, and wherein the training comprises:
receiving a plurality of first training inputs, and, for each first training input:
processing the first training input using the VAE to determine a latent representation that includes a plurality of latent factors and to generate a reconstruction of the first training input in accordance with current values of the parameters of the VAE; and
adjusting current values of the parameters of the VAE by optimizing a training objective function that depends on a quality of the reconstruction and also on a degree of independence between the latent factors in the latent representation of the first training input.

7. The method of claim 6, wherein some or all of the plurality of first training inputs are unlabeled EEG training inputs.

8. A system comprising one or more computers and one or more storage devices storing instructions that when executed by the one or more computers cause the one or more computers to perform operations for training an auto-encoder to de-noise task specific electroencephalogram (EEG) signals, the operations comprising:
training a variational auto-encoder (VAE) including to learn a plurality of parameter values of the VAE by applying, as first training input to the VAE, training data, the training data comprising electroencephalogram (EEG) data representing brain activities of individual persons when performing different tasks, wherein the VAE is configured to receive as input EEG data, process the EEG data to determine a latent representation of the EEG data, and to process the latent representation to generate a reconstruction of the EEG data; and
after the training, adapting the VAE for a specific task by applying, as second training input to the VAE, adaptation data, the adaptation data comprising task-specific EEG data representing brain activities of individual persons when performing the specific task, wherein adapting the VAE comprises adjusting learned parameter values of the VAE by optimizing an adaptation objective function that depends at least on a quality of the reconstructions of the task-specific EEG data.

9. The system of claim 8, wherein adapting the VAE for the specific task comprises:
training the VAE using a de-noising auto-encoder training technique.

10. The system of claim 8, wherein the operations further comprise, after the adaptation:
providing data specifying the trained VAE for deployment in an EEG diagnosis system to determine mental health conditions of individual persons based on processing EEG data.

11. The system of claim 10, wherein the EEG diagnosis system comprises one or more classification models or one or more prediction models.

12. The system of claim 8, wherein the VAE is a convolutional disentangling VAE, and wherein the training comprises:
receiving a plurality of first training inputs, and, for each first training input:
processing the first training input using the VAE to determine a latent representation that includes a plurality of latent factors and to generate a reconstruction of the first training input in accordance with current values of the parameters of the VAE; and
adjusting current values of the parameters of the VAE by optimizing a training objective function that depends on a quality of the reconstruction and also on a degree of independence between the latent factors in the latent representation of the first training input.

13. The system of claim 12, wherein some or all of the plurality of first training inputs are unlabeled EEG training inputs.

14. One or more non-transitory computer-readable storage media storing instructions that when executed by one or more computers cause the one or more computers to perform operations for training an auto-encoder to de-noise task specific electroencephalogram (EEG) signals, the operations comprising:
training a variational auto-encoder (VAE) including to learn a plurality of parameter values of the VAE by applying, as first training input to the VAE, training data, the training data comprising electroencephalogram (EEG) data representing brain activities of individual persons when performing different tasks, wherein the VAE is configured to receive as input EEG data, process the EEG data to determine a latent representation of the EEG data, and to process the latent representation to generate a reconstruction of the EEG data; and
after the training, adapting the VAE for a specific task by applying, as second training input to the VAE, adaptation data, the adaptation data comprising task-specific EEG data representing brain activities of individual persons when performing the specific task, wherein adapting the VAE comprises adjusting learned parameter values of the VAE by optimizing an adaptation objective function that depends at least on a quality of the reconstructions of the task-specific EEG data.

15. The non-transitory computer-readable storage media of claim 14, wherein adapting the VAE for the specific task comprises:
training the VAE using a de-noising auto-encoder training technique.

16. The non-transitory computer-readable storage media of claim 14, wherein the operations further comprise, after the adaptation:
providing data specifying the trained VAE for deployment in an EEG diagnosis system to determine mental health conditions of individual persons based on processing EEG data.

17. The non-transitory computer-readable storage media of claim 14, wherein the VAE is a convolutional disentangling VAE, and wherein the training comprises:
receiving a plurality of first training inputs, and, for each first training input:
processing the first training input using the VAE to determine a latent representation that includes a plurality of latent factors and to generate a reconstruction of the first training input in accordance with current values of the parameters of the VAE; and
adjusting current values of the parameters of the VAE by optimizing a training objective function that depends on a quality of the reconstruction and also on a degree of independence between the latent factors in the latent representation of the first training input.

18. An electroencephalogram (EEG) de-noising and diagnosis system comprising:
an array of variational auto-encoders (VAE), wherein each VAE is trained by applying, as first training input to the VAE, training data comprising EEG data representing brain activities of individual persons when performing different tasks, and, after being trained, each VAE is adapted for a specific task by applying, as second training input to the VAE, adaptation data, the adaptation data comprising task-specific EEG data representing brain activities of individual persons when performing the specific task, wherein the array comprises:
a first VAE that is adapted to de-noise EEG data associated with a first type of task; and
a second VAE that is adapted to de-noise EEG data associated with a second, different type of task.

19. The EEG de-noising and diagnosis system of claim 18, wherein the system further comprises one or more classification models or one or more prediction models.

20. The EEG de-noising and diagnosis system of claim 18, wherein the specific task comprises a reinforcement learning reward task.
